(19) United States
(12) Patent Application Publication
(10) Pub. No.: US 2011/0263025 A1
(43) Pub. Date: Oct. 27, 2011

(54) BIODEGRADABLE POLYDISULFIDE AMINES FOR GENE DELIVERY

(75) Inventors: Mei Ou, Lansdale, PA (US); Sung Wan Kim, Salt Lake City, UT (US)

(73) Assignee: UNIVERSITY OF UTAH RESEARCH FOUNDATION, Salt Lake City, UT (US)

(21) Appl. No.: 13/132,210

(22) PCT Filed: Dec. 2, 2009
(86) PCT No.: PCT/US09/66443

§ 371 Date: Jun. 1, 2011

Related U.S. Application Data
(62) Division of application No. 61/119,281, filed on Dec. 2, 2008.
Application No. 61/119,281 is a division of application No. 61/210,514, filed on Mar. 19, 2009.

(51) Int. Cl. C12N 15/85; C07H 21/02; C07H 21/00; C12N 15/63; C12N 5/071; C07C 323/41

(52) U.S. Cl. 435/455; 435/375; 564/153; 536/23.1; 435/320.1; 536/24.5

(57) ABSTRACT

Poly(disulfide amine)s, methods of making, and methods of use are described. Illustrative embodiments of the poly(disulfide amine)s include poly(N,N′-cystaminebisacrylamide-spermine), poly(N,N′-cystaminebisaciylamide-N,N′-bis(3-aminopropyl)1,3-propanediamine), poly(N,N′-cystaminebisacrylamide-N,N′-bis(3-amino-propyl)ethylenediamine), poly(N,N′-cystaminebisacrylamide-N,N′-bis(2-aminoethyl)-1,3-propanediamine), and poly(N,N′-cystaminebisacrylamide-triethylenetetramine). These compositions are made by Michael addition between N,N′-cystaminebisacrylamide and protected oligoamine monomers, followed by deprotection. Complexes are formed by mixing the poly(disulfide amine)s with a nucleic acid. Delivery of the nucleic acid into cells is carried out by contacting the cells with the nucleic acid/poly(disulfide amine) complexes.

This invention relates to gene delivery. More particularly, this invention relates to nonviral gene delivery carriers.

Gene therapy has broad potential in treatment of human genetic and acquired diseases through the delivery and application of therapeutic gene-based drugs. The use of safe, efficient and controllable gene carriers is a requirement for the success of clinical gene therapy. R. C. Mulligan, The basic science of gene therapy, 260 Science 926-932 (1993); I. M. Verma & N. Somia, Gene therapy—promises, problems and prospects, 389 Nature 239-242 (1997). Although viral vectors are very efficient in gene delivery, their potential safety and immunogenicity concerns raise their risk in clinical applications. C. Baum et al., Mutagenesis and oncogenesis by chromosomal insertion of gene transfer vectors, 17 Hum. Gene Ther. 253-263 (2006). As an alternative to viral vectors, cationic polymers such as poly(L-lysine) (PLL), poly(ethylenimine) (PEI), poly(amidoamine) dendrimers, and cationic liposomes, have been synthesized as gene delivery carriers. The advantages of these cationic polymer carriers include safety, stability, large DNA and RNA loading capacity, and easy and large-scale production. S. Li & L. Huang, Nonviral gene therapy: promises and challenges, 7 Gene Ther. 31-34 (2000); F. Liu et al., Non-immunostimulatory nonviral vectors, 18 Faseb J. 1779-1781 (2004); T. Niidome & L. Huang, Gene therapy progress and prospects: nonviral vectors, 9 Gene Ther. 1647-1652 (2002). The cationic polymers can condense negatively charged DNA into nanosized particles through electrostatic interactions, and the polymer/plasmid DNA (pDNA) polyplexes can enter cells via endocytosis. Y. W. Cho et al., Polycation gene delivery systems: escape from endosomes to cytosol, 55 J. Pharm. Pharmacol. 721-734 (2003); L. De Laporte et al., Design of modular non-viral gene therapy vectors, 27 Biomaterials 947-954 (2006); E. Piskin et al., Gene delivery: intelligent but just at the beginning, 15 J. Biomater. Sci. Polym. Ed. 1182-1202 (2004). As a result, the polymers can protect pDNA from nuclease degradation, and facilitate cellular uptake to induce high gene transfection. O. Boussif et al., A versatile vector for gene and oligonucleotide transfer into cells in culture and in vivo: polyethylenimine, 92 Proc. Nat'l Acad. Sci. USA 7297-7301 (1995); D. W. Pack et al., Design and development of polymers for gene delivery, 4 Nat. Rev. Drug. Discov. 581-593 (2005).

An illustrative embodiment of the present invention comprises a composition comprising a poly(disulfide amine). Illustrative examples of poly(disulfide amine)s according to the present invention comprise poly(CBA-SP), poly (CBA-APPD), poly(CBA-APED), poly(CBA-AEPD), and poly(CBA-TETA).

Another illustrative embodiment of the present invention comprises a complex comprising a selected nucleic acid bonded to a poly(disulfide amine). The bonding of the nucleic acid to the poly(disulfide amine) is typically by electrostatic interactions of the negatively charged nucleic acid to the positively charged poly(disulfide amine). Illustrative poly(disulfide amine)s according to this embodiment of the present invention comprise poly(CBA-SP), poly (CBA-APPD), poly(CBA-APED), poly(CBA-AEPD), and poly(CBA-TETA). Illustrative nucleic acids comprise plasmids, siRNA (small interfering RNA), oligonucleotides, and other DNAs and/or RNAs.

Still another illustrative embodiment of the present invention comprises a method for transfecting mammalian cells, the method comprising contacting selected mammalian cells with a complex comprising a nucleic acid bonded to a poly(disulfide amine). Illustrative poly(disulfide amine)s comprise poly(CBA-SP), (CBA-APPD), poly(CBA-APED), poly(CBA-AEPD), and poly(CBA-TETA). Illustrative nucleic acids comprise plasmids, siRNA, oligonucleotides, and other DNAs and/or RNAs.

Another illustrative embodiment of the invention comprises a method comprising contacting selected mammalian cells with a complex comprising a nucleic acid bonded to a poly(disulfide amine), wherein the poly(disulfide amine) is selected from poly(CBA-SP), poly(CBA-APPD), poly(CBA-APED), poly(CBA-AEPD), poly(CBA-TETA), and mixtures thereof.

Yet another illustrative embodiment of the present invention comprises a method for making a poly(disulfide amine), the method comprising:

(a) reacting N,N′-cystaminebisacrylamide and a primary-amine-protected oligoamine monomer to result in a primary-amine-protected poly(disulfide amine); and

(b) deprotecting the primary-amine-protected poly(disulfide amine) to result in the poly(disulfide amine).

According to this illustrative embodiment, the primary-amine-protected oligoamine monomer may comprise Dde-protected SP, Dde-protected APPD, Dde-protected APED, Dde-protected AEPD, or Dde-protected TETA, and the poly(disulfide amine)s may comprise poly(CBA-SP), poly(CBA-APPD), poly(CBA-APED), poly(CBA-AEPD), or poly(CBA-TETA).

### BRIEF DESCRIPTION OF THE SEVERAL VIEWS OF THE DRAWINGS

FIG. 1 shows a scheme for synthesis of poly(disulfide amine)s according to the present invention.

FIGS. 2A-E show ¹H NMR spectra for poly(CBA-SP), poly (CBA-APPD), poly(CBA-APED), poly(CBA-AEPD), and poly(CBA-TETA), respectively.

FIGS. 3A-E show FPLC data for poly(CBA-SP), poly (CBA-APPD), poly(CBA-APED), poly(CBA-AEPD), and poly(CBA-TETA), respectively.

FIG. 4 shows titration curves obtained by titrating aqueous poly(disulfide amine)s (5 mM amino nitrogen atoms) in 10 mL of 1.0 M aqueous NaCl. Solutions were set initially at pH 11.0 with 0.1 M NaOH and then were titrated with 0.01 M HCl. As references, the titration curves of bPEI 25 kDa and 0.1 M NaCl were also determined.

FIG. 5 shows average particle sizes of poly(disulfide amine)/pDNA complexes and control bPEI 25 kDa/pDNA complexes measured at w/w ratios of 1, 5, 10, 20, and 30.

FIGS. 6A and 6B show agarose gel electrophoresis of poly(disulfide amine)/pDNA polyplexes at different w/w ratios in the absence (FIG. 6A) and presence (FIG. 6B) of 10.0 mM dithiothreitol (DTT, 37° C., 1 h): lane 1, naked plasmid DNA; lane 2, bPEI/pDNA at w/w ratio of 1:1; lanes 3-11, poly(disulfide amine)/pDNA at w/w ratios of 0.1, 0.2, 0.5, 1, 2, 5, 10, 20, and 30, respectively.

FIGS. 7A-B show transfection efficiencies of poly(disulfide amine)/pDNA complexes at w/w ratios of 1, 5, 10, 20, and 30; Control—non-treated cells; Positive control: bPEG 25 kDa at w/w ratio of 0.6:1. Results are expressed as relative luminescence units (RLU) of luciferase reporter gene expression normalized for total cell protein content in each well as mean values of triplicate samples±standard deviations in HeLa (human cervical cancer) cells (FIG. 7A) and C2C12 (mouse myoblast) cells (FIG. 7B).

FIG. 8 shows relative cell viabilities of poly(disulfide amine)/pDNA polyplexes and bPEG 25 kDa/pDNA control polyplexes in C2C12 cells at w/w ratios of 1, 5, 10, 20, and 30 compared to an untreated control group. Cytotoxicity was determined by MTT assay, and data points are means of triplicate samples±standard deviations.

FIGS. 9A-E show the cellular uptake of poly(disulfide amine)s/DNA polyplexes in C2C12 cells: poly (CBA-SP), FIG. 9A; poly (CBA-APPD), FIG. 9B; poly (CBA-APED), FIG. 9C; poly (CBA-AEPD), FIG. 9D; poly (CBA-TETA), FIG. 9E. Fluorescence histogram intensities correspond to polymer/DNA w/w ratios and are represented as control, untreated cells (100); 1:1 (102); 5:1 (104); 10:1 (106); 20:1 (108); 30:1 (110); M1 region (M1 gated fluorescence intensity).

### DETAILED DESCRIPTION

Before the present nonviral gene delivery carriers and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

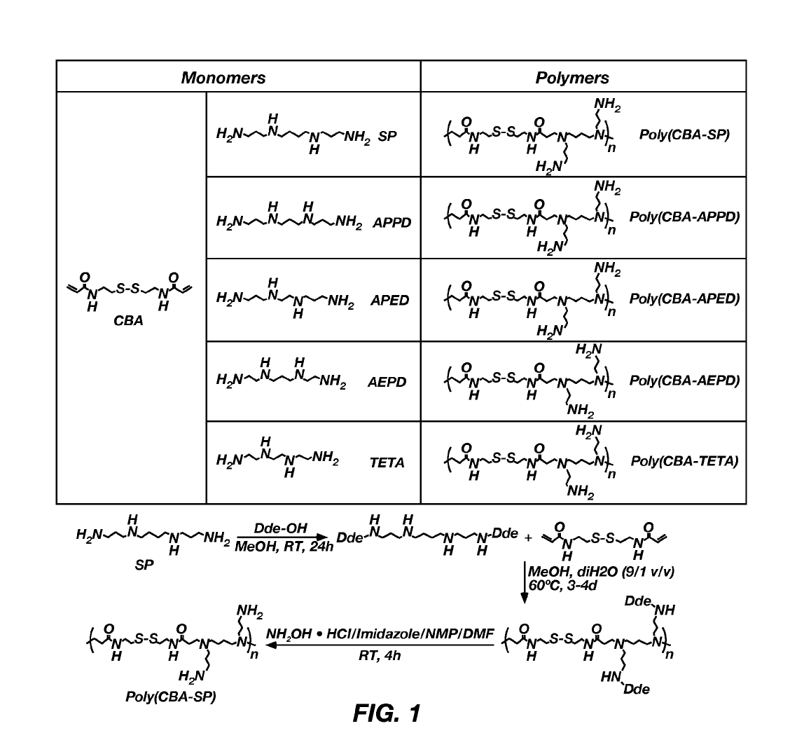

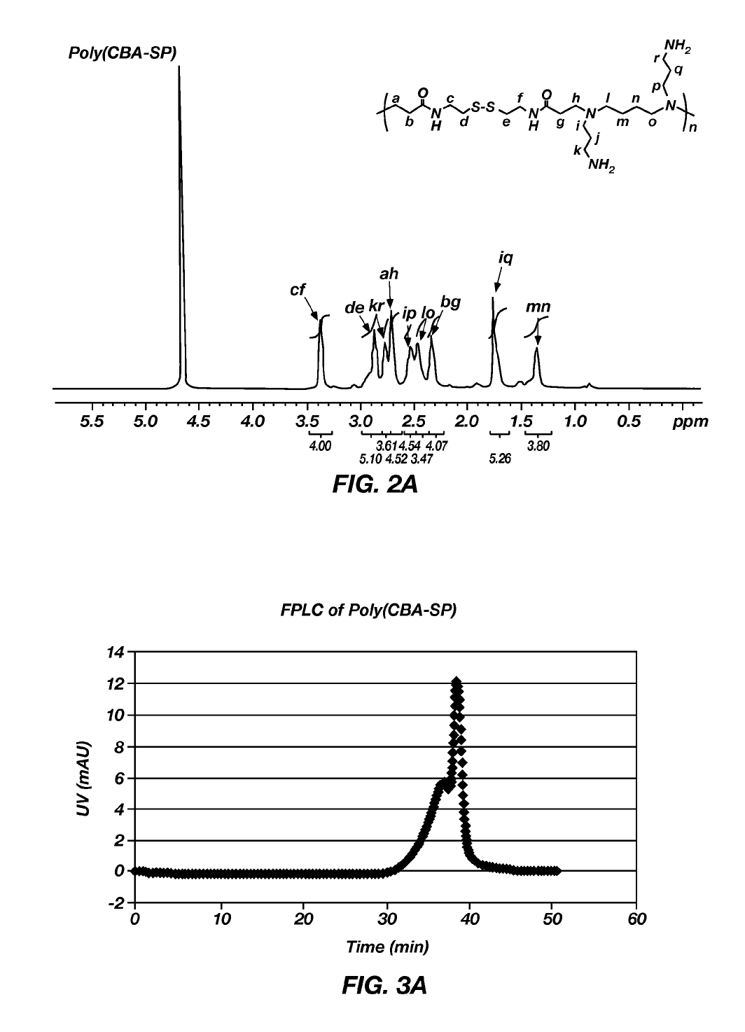

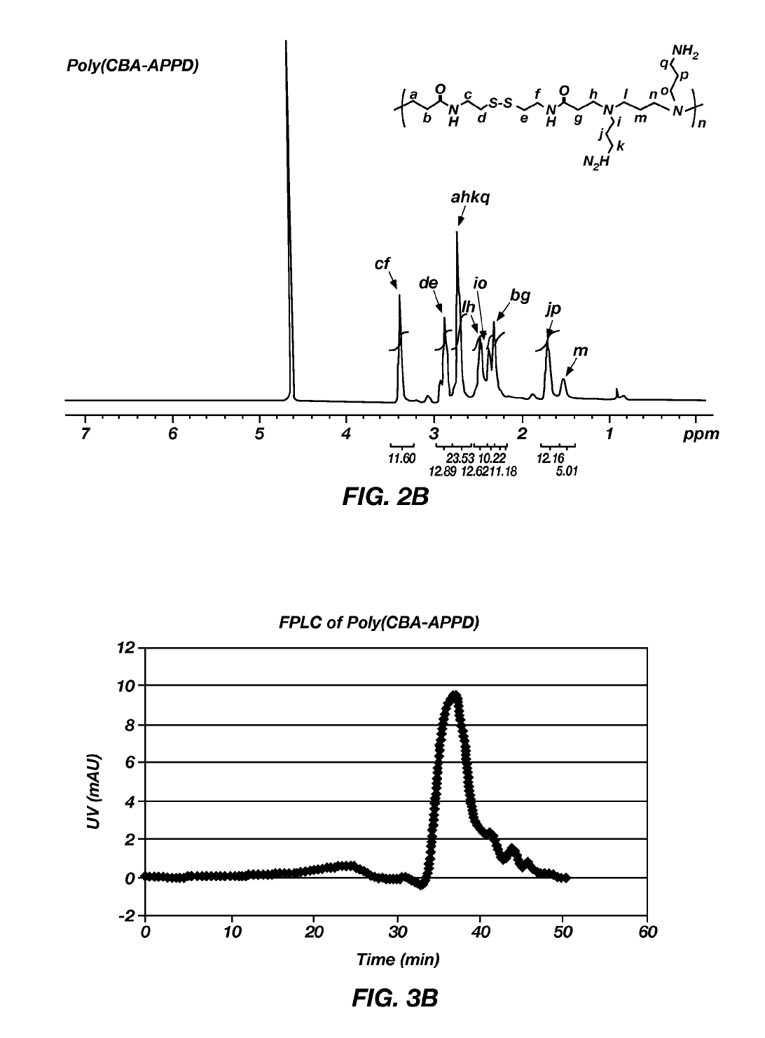

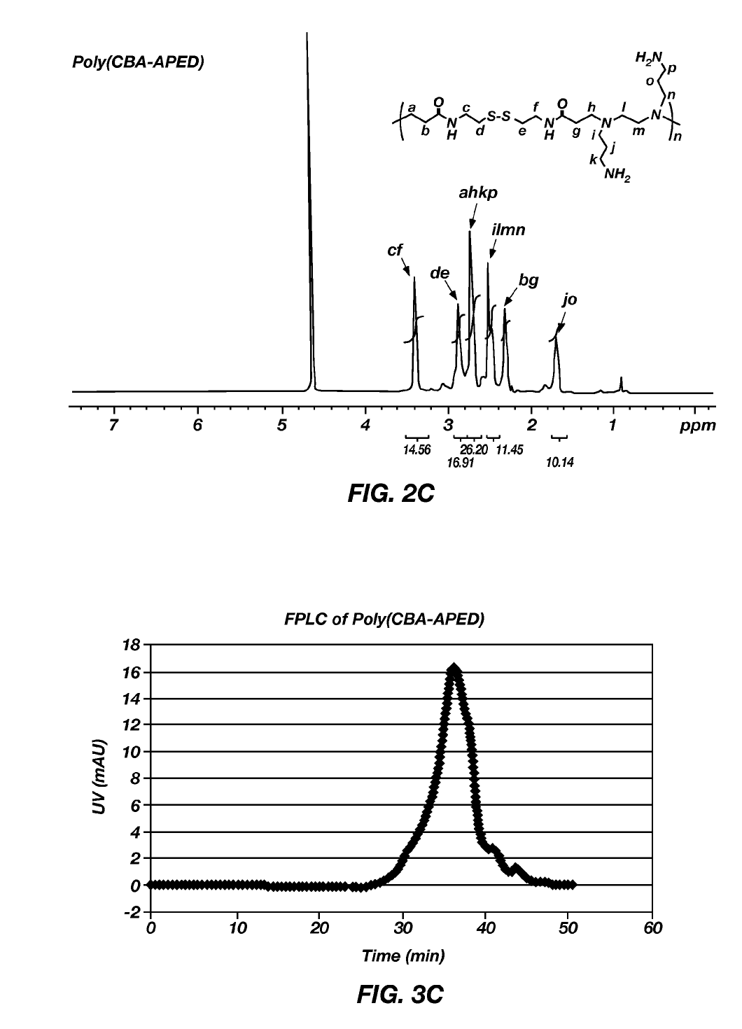

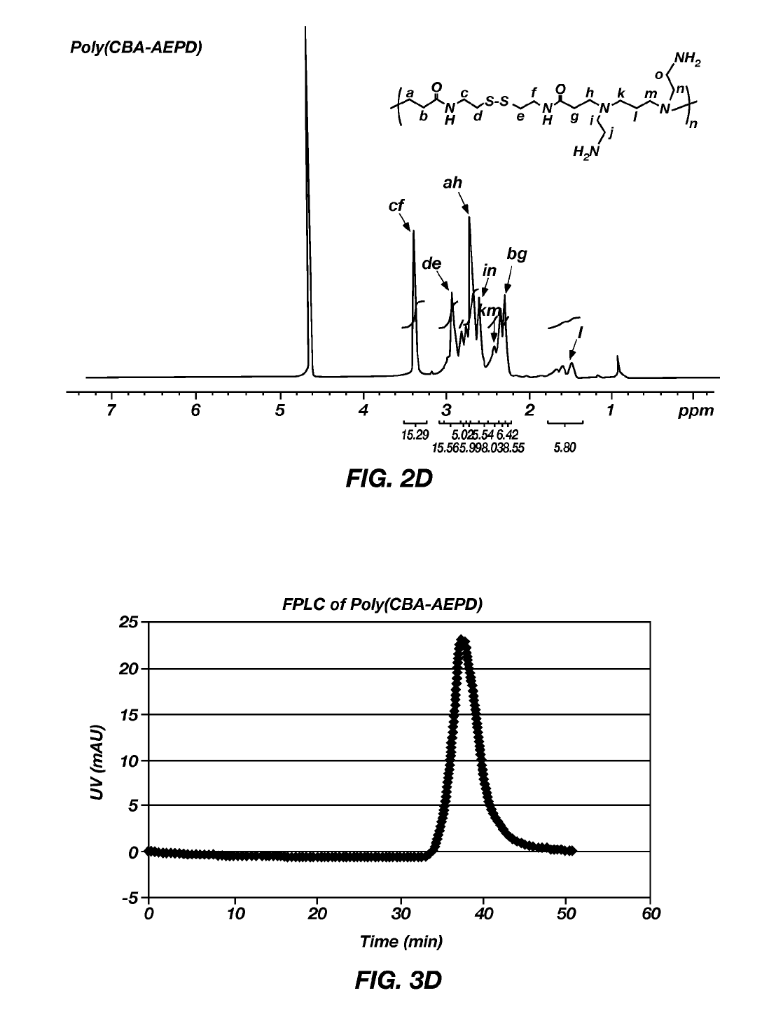

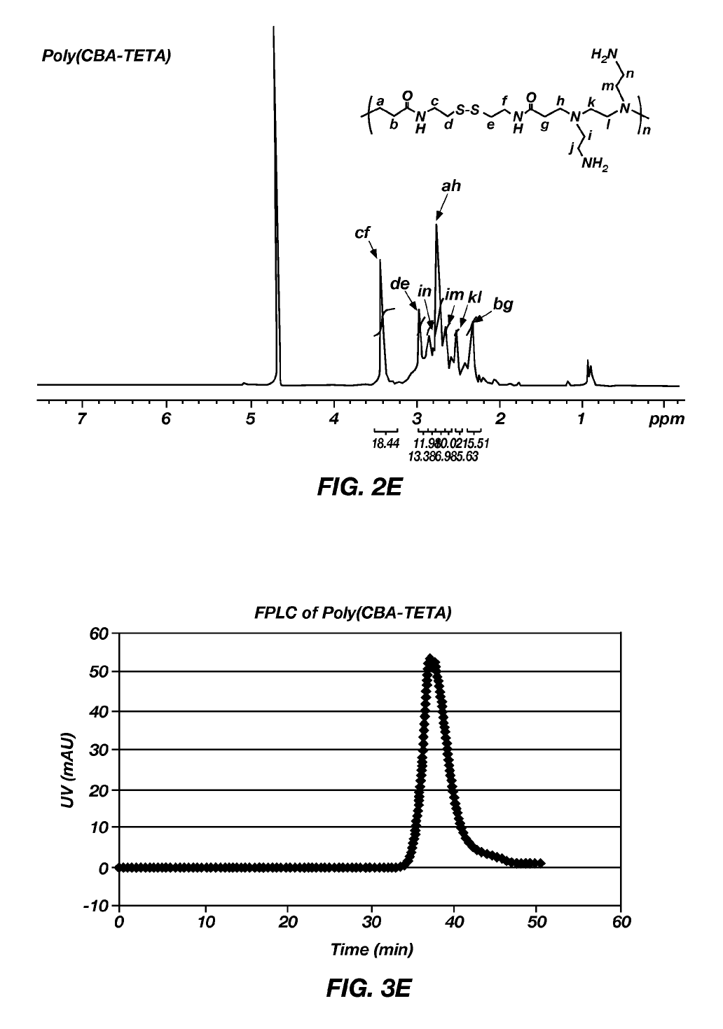

As used herein, “poly(CBA-SP)” means poly(N,N′-cystaminebisacrylamide-spermine), as illustrated in FIG. 1 and FIG. 2A.

As used herein, “poly(CBA-APPD)” means poly(N,N′-cystaminebisacrylamide-N,N′-bis(3-aminopropyl)-1,3-propanediamine), as illustrated in FIG. 1 and FIG. 2B.

As used herein, “poly(CBA-APED)” means poly(N,N′-cystaminebisacrylamide-N,N′-bis(3-aminopropyl)ethylenediamine), as illustrated in FIG. 1 and FIG. 2C.

As used herein, “poly(CBA-AEPD)” means poly(N,N′-cystaminebisacrylamide-N,N′-bis(2-aminoethyl)-1,3-propanediamine), as illustrated in FIG. 1 and FIG. 2D.

As used herein, “poly(CBA-TETA)” means poly(N,N′-cystaminebisacrylamide-triethylenetetramine), as illustrated in FIG. 1 and FIG. 2E.

As used herein, “pDNA” means plasmid DNA and “bPEG 25 kDa” means branched polyethylene glycol having a nominal molecular weight of about 25,000.

As used herein, “comprising,” “including,” “containing,” and grammatical equivalents thereof are inclusive or open-ended terms that do not exclude additional, unrecited elements or method steps.

A group of bioreducible poly(disulfide amine)s were synthesized and characterized as non-viral gene carriers with defined structure, high transfection efficiency, and low cytotoxicity. First, the primary amine groups of five oligoamines, spermine (SP); N,N′-bis(3-aminopropyl)-1,3-propanediamine (APPD); N,N′-bis(3-aminopropyl)ethylenediamine (APED); N,N′-bis(2-aminoethyl)-1,3-propanediamine (AEPD); and triethylenetetramine (TETA), were protected by 2-acetyldimedone (Dde-OH). Second, polymers were synthesized by Michael addition between N,N′-cystaminebisacrylamide (CBA) and the five Dde-protected oligoamines. After deprotecting the Dde-group with NH₂OH.HCl/Imidazole/NMP/DMF solution, five linear and bioreducible poly(disulfide amine)s, poly(CBA-SP), poly(CBA-APPD), poly(CBA-APED), poly(CBA-AEPD) and poly(CBA-TETA), were synthesized with disulfide bonds and tertiary amine groups in their main chain and pendant primary amine groups in side chains. Polymer structures were confirmed by ¹H NMR, and their weight average molecular weights, determined by size exclusion chromatography (SEC), were in the range of 3.8˜6.1 kDa with narrow polydispersity (1.15˜1.33). Acid-base titration assay showed that the five poly(disulfide amine)s possessed superior buffering capacity to branched PEI 25 kDa in the pH range of 7.4˜5.1. All these poly(disulfide amine)s can efficiently condense plasmid DNA into nanosized particles (<200 nm). Agarose gel electrophoresis demonstrated that poly(disulfide amine)s can completely condense plasmid DNA from w/w ratio of 2:1 and above. With the incubation of 10.0 mM DTT for 1 h, significant polyplexes dissociation was observed due to the cleavage of disulfide bonds, mimicking the intracellular reduction conditions. In vitro transfection experiments with Hela and C2C12 cells showed that the five polyplexes have superior luciferase expression than bPEI 25 kDa from w/w ratios of 5 to 30. In addition, poly(CBA-SP), poly(CBA-APPD) and poly(CBA-APED) have higher transfection efficiencies than poly(CBA-AEPD) and poly(CBA-TETA). Furthermore, MTT assay indicated that all five poly(disulfide amine)/pDNA polyplexes were significantly less toxic than bPEI/pDNA complexes.

Synthesis and characterization of bioreducible poly(disulfide amine)s. A group of bioreducible poly(disulfide amine)s were synthesized and characterized as non-viral gene carriers with defined structure, high transfection efficiency and low cytotoxicity. First, the primary amine groups of five oligoamines, spermine (SP), N,N′-bis(3-aminopropyl)-1,3-propanediamine (APPD), N,N′-bis(3-aminopropyl)ethylenediamine, (APED), N,N′-bis(2-aminoethyl)-1,3-propanediamine (AEPD), and triethylenetetramine (TETA), were protected by 2-acetyldimedone (Dde-OH). Second, polymers were synthesized by Michael addition between N,N′-cystaminebisacrylamide (CBA) and the five Dde-protected oligoamines. After deprotecting Dde-groups with NH₂OH.HCl/Imidazole/NMP/DMF solution, five linear and bioreducible poly(disulfide amine)s, poly(CBA-SP), poly(CBA-APPD), poly(CBA-APED), poly(CBA-AEPD) and poly(CBA-TETA) were synthesized. These poly(disulfide amine)s contain one disulfide bond and two tertiary amine groups in their main chain and two pendant primary amine groups in side chains in each repeating units (FIG. 1). All poly(disulfide amine)s were purified by dialysis and lyophilized as gel products and were readily soluble in water, PBS buffer, HEPES buffer, Tris buffer, dimethyl sulfoxide (DMSO) and methanol. The final structures of poly(disulfide amine)s were confirmed by ¹H NMR (400 MHZ, D₂O) (FIGS. 2A-E). The disappearance of signal peaks between δ 5 to 7 ppm indicated that the polymerization was complete and the acrylamide end groups no longer existed in the final polymer products. The final polymers have defined structures without any branches formation during the synthesis.

The molecular weights of these polymers were measured by fast protein liquid chromatography (FPLC; FIGS. 3A-E) and calibrated with pHPMA standards (Table 1). The range of the weight average molecular weight (M_{w}) of these polymers was from 3.8˜6.1 kDa, while the range of the number average molecular weight (Mₙ) was from 3.2˜4.6 kDa. The polydispersity index (PDI), ranging from 1.15˜1.33, indicates that these poly(disulfide amine)s have a narrow molecular weight distribution.

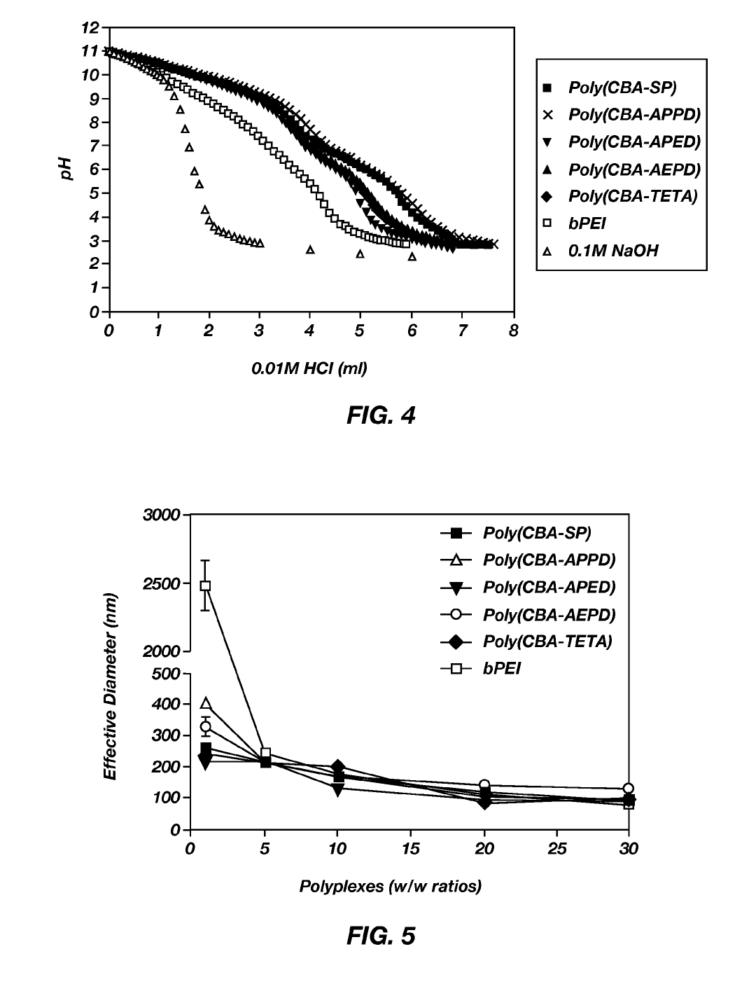

Buffering capacity is an important factor for cationic gene carriers, measured by acid-base titration, were expressed as the percentage of amine groups becoming protonated from pH 7.4 to 5.1, mimicking the change from the high pH extracellular environment to the low pH endosomal environment. The results (FIG. 4) show that all five poly(disulfide amine)s have excellent buffering capacity, which is 38%, 36%, 26%, 28% and 28% protonation for poly(CBA-SP), poly(CBA-APPD), poly(CBA-APED), poly(CBA-AEPD) and poly(CBA-TETA), respectively. In comparison, bPEI 25 kDa has lower buffering capacity (22% protonation) under the same conditions. The high buffering capacities enable poly(disulfide amine)s to facilitate plasmid DNA endosomal escape, contributing to efficient gene transfection.

DNA condensation and release. Dynamic light scattering (DLS) studies showed that these five poly(disulfide amine)s can condense plasmid DNA to nanosized particles with effective diameters less than 200 nm at polymer/pDNA w/w ratios of 5:1 and above via electrostatic interactions between the positive charged polymers and the negative charged phosphates on DNA backbones (FIG. 5).

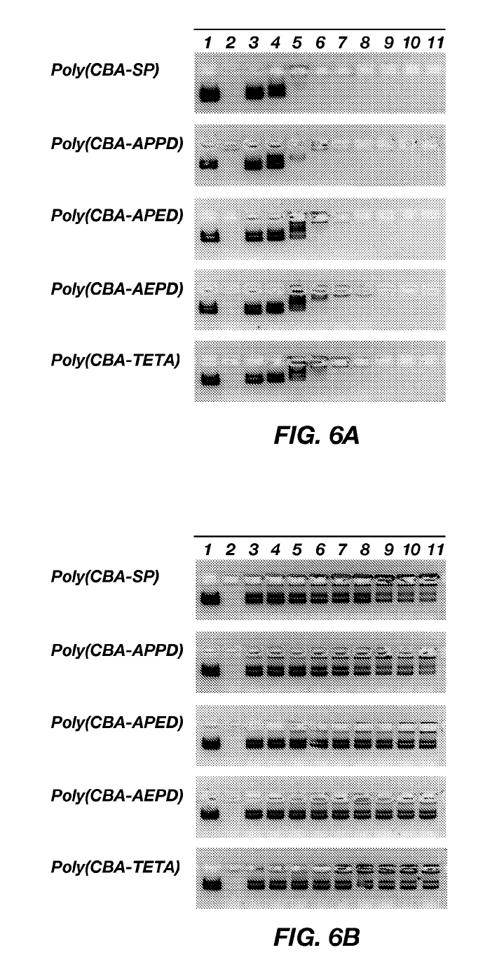

Agarose gel electrophoresis further verified that poly(disulfide amine)s can condense plasmid DNA at low w/w ratios (FIGS. 6A and 6B). In the absence of DTT incubation (FIG. 6A), poly(CBA-SP), poly(CBA-APPD), poly(CBA-APED) can completely retard plasmid DNA from w/w ratio of 1:1, while poly(CBA-AEPD) and poly(CBA-TETA) can completely condense plasmid DNA from w/w ratios of 2:1. When the polyplexes were incubated with 10.0 mM DTT at 37° C. for 1 hr, plasmid DNA was released from all poly(disulfide amine)s at all w/w ratios (FIG. 6B). For the non-degradable polymer bPEI 25 kDa, there was no pDNA released from polyplexes in the presence of DTT. Therefore, all poly(disulfide amine)s can release pDNA efficiently via disulfide bonds cleavage, leading to increased DNA release and increased gene expression.

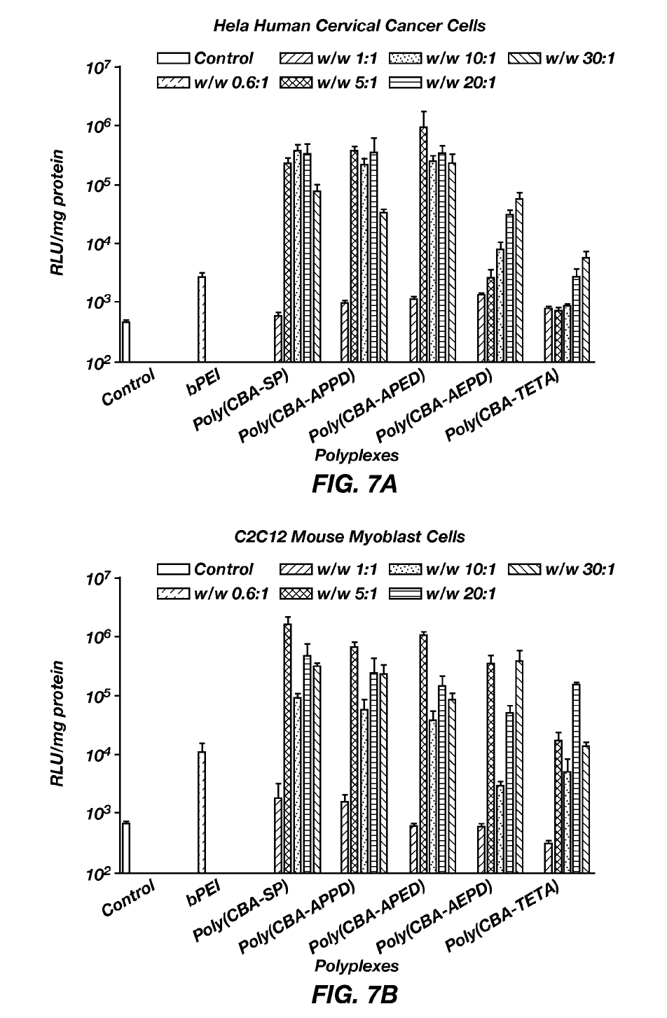

In vitro transfection efficiency and cytotoxicity. In vitro transfection efficiency of these bioreducible poly(disulfide amine)s were evaluated by luciferase assay, using reporter gene pCMV-Luc (1.0 μg/mL) on Hela and C2C12 cells, at w/w ratios of 1, 5, 10, 20, and 30 in the absence of serum (FIGS. 7A-B). Complexes of bPEI (25 kDa)/pDNA at w/w ratio of 0.6:1 were used as a positive control, which is about N/P ratio of 5:1. At this w/w ratio, bPEI showed the highest gene transfection efficiency and low cytotoxicity. The transfection efficiency was quantitatively measured as luciferase enzyme activity and normalized as total cell protein concentration by BCA protein assay. All these poly(disulfide amine)s showed relatively higher gene transfection efficiency than bPEI in both cell lines from w/w ratio of 5 to 30. Among these poly(disulfide amine)s, poly(CBA-SP), poly(CBA-APPD) and poly(CBA-APED) showed higher levels of gene expression than poly(CBA-AEPD) and poly(CBA-TETA).

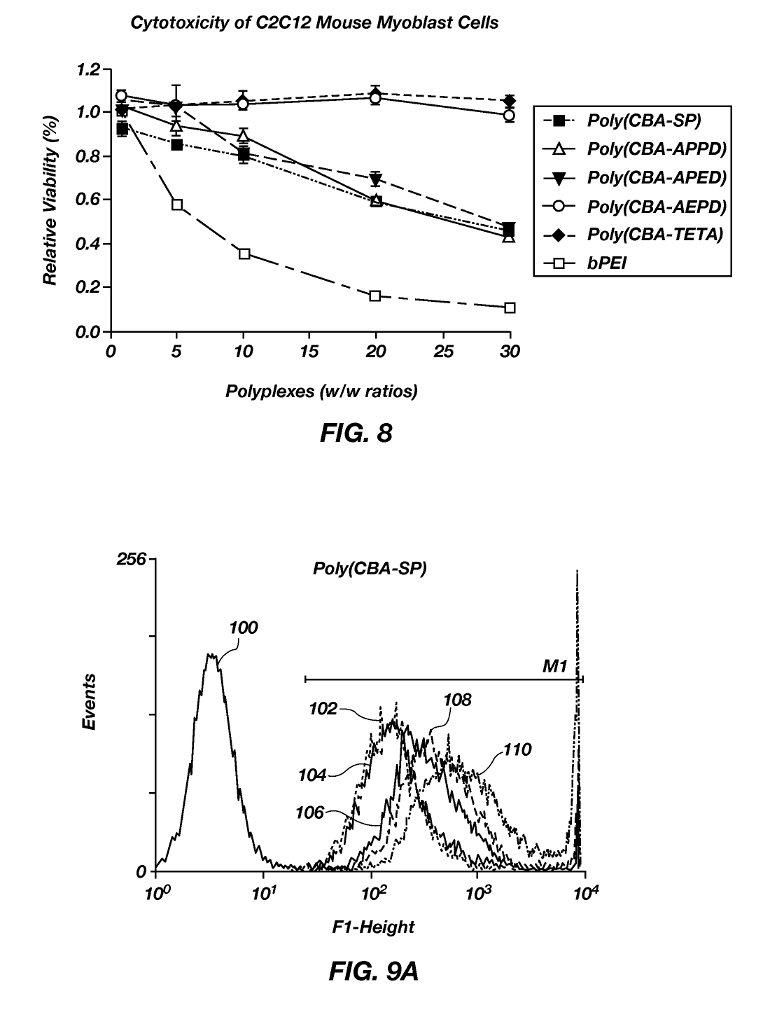

In vitro cytotoxicity of poly(disulfide amine)s was evaluated by MTT assay on C2C12 cells (FIG. 8). The experiments were performed the same manner as the transfection experiments described above, except that MTT assay was performed at 24 hrs instead of 48 hrs post-transfection. As expected, poly(disulfide amine)s showed much lower cytotoxicity than bPEI 25 kDa. The overall profile in FIG. 8 showed that bPEI 25 kDa has increasing cytotoxicity from w/w ration of 5 and above. In contrast, all poly(disulfide amine)s showed much lower toxicity. Further, poly(CBA-AEPD) and poly(CBA-TETA) showed much lower toxicity than poly(CBA-SP), poly(CBA-APPD) and poly(CBA-APED). In summary, the family of bioreducible poly(disulfide amine)s have high gene transfection efficiency and low cytotoxicity, which are advantageous for gene delivery.

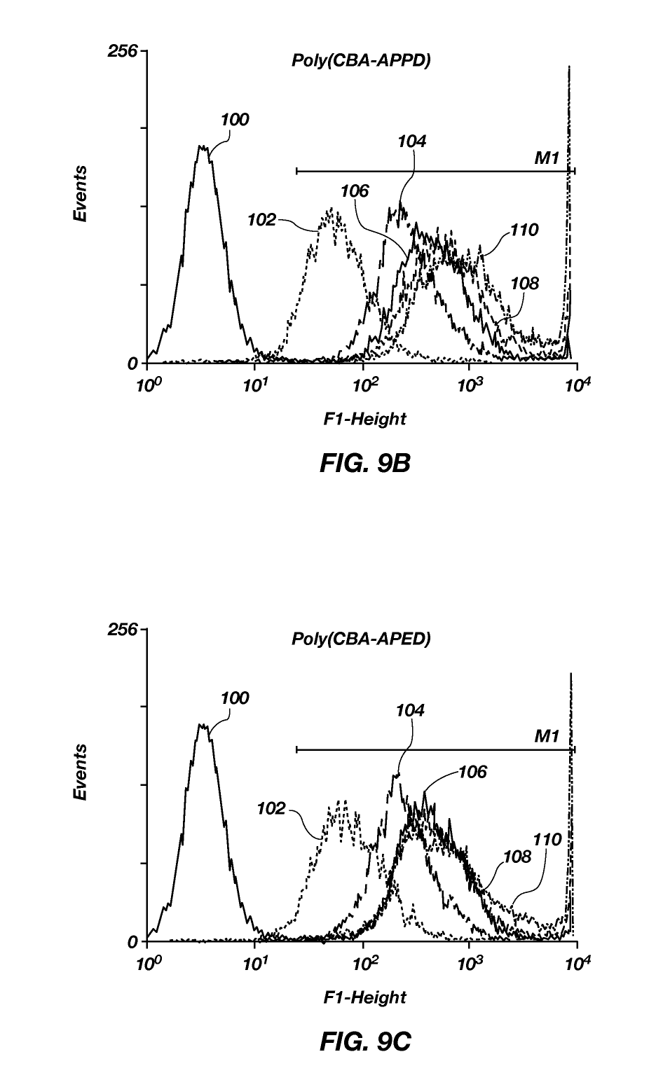

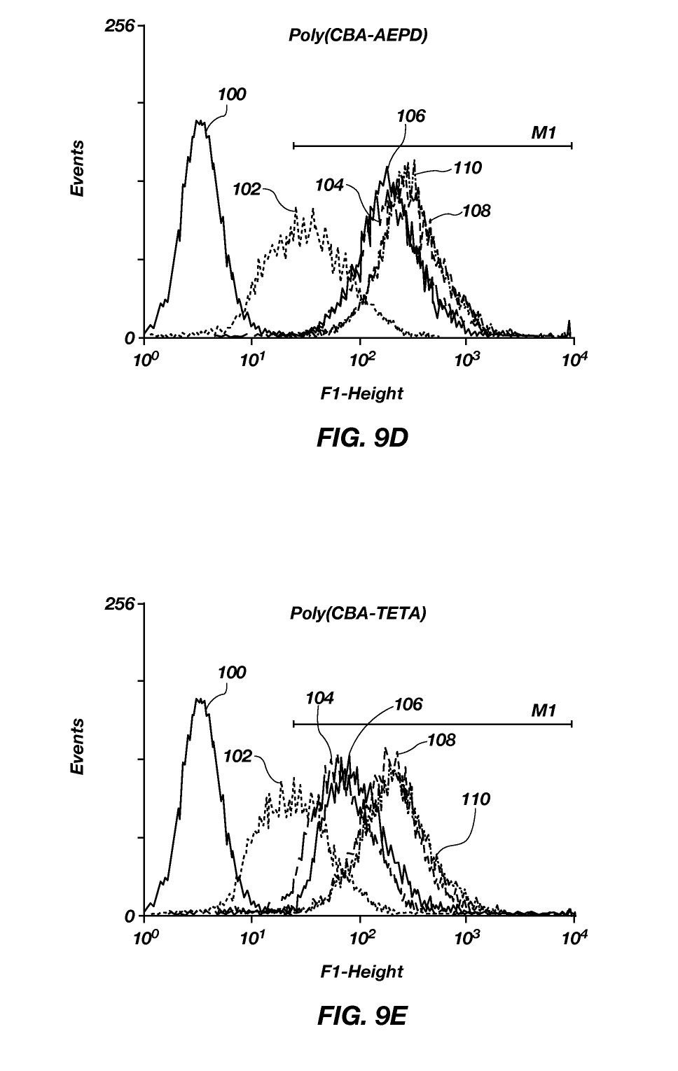

Cellular uptake. Using YOYO-1 iodide-intercalated pCMV-Luc, the cellular uptake of the polyplexes was estimated by flow cytometry in C2C12 cells as a function of the poly(disulfide amine)s/DNA w/w ratios (FIGS. 9A-E). The results showed that the uptake of polyplexes increased with the increase of w/w ratios from 1:1 to 30:1, with the fluorescent signals shifting to the stronger histogram area (labeled “M1”). The proportion of cells taking up polyplexes (% cell count in the R2 region) by poly(CBA-SP), poly(CBA-APPD), and poly(CBA-APED) was about 99% at all w/w ratios. At a w/w ratio of 1:1, poly(CBA-AEPD) and poly(CBA-TETA) induced 77% and 71% cellular uptake, respectively. At w/w ratios of 5:1 and above, poly(CBA-AEPD) and poly1(CBA-TETA) increased cellular uptake to about 99%. These results agreed with previous results that poly(disulfide amine)s can mediate high levels of gene transfection in cells. Also, poly (CBA-SP), poly(CBA-APPD), and poly(CBA-APED), which contain the polypropylene side spacers [—(CH₂)₃—], can induce higher levels of cellular uptake and transfection efficiency than poly(CBA-AEPD) and poly(CBA-TETA), which contain the ethylene side spacers [—(CH₂)₂—].

### EXAMPLES

### Materials

All chemicals, spermine (SP, Sigma, St. Louis, Mo.); N,N′-bis(3-aminopropyl)-1,3-propanediamine (APPD, Sigma-Aldrich, St. Louis, Mo.); N,N′-bis(3-aminopropyl)-ethylenediamine, (APED, Acros Organics, Fair Lawn, N.J.); N,N′-bis(2-aminoethyl)-1,3-propanediamine (AEPD, Sigma-Aldrich); triethylenetetramine (TETA, Sigma-Fluka, St. Louis, Mo.); N,N′-cystaminebisacrylamide (CBA, PolySciences, Warrington, Pa.); hyperbranched polyethylenimine (bPEI, M_{w}=25 kDa, Sigma); ethylenediamine (EDA, Sigma-Aldrich); 2-acetyldimedone (Dde-OH, EMD Chemicals, Inc. Gibbstown, N.J.); hydroxylamine hydrochloride (NH₂OH.HCl, Sigma-Aldrich); imidazole (Sigma-Aldrich); N-methyl-2-pyrrolidinone (NMP, Sigma-Aldrich); N,N-dimethylformamide (DMF, Sigma-Aldrich); 3-(4,5-dimethyl-2-thiazolyl)-2,5-diphenyl-2H-tetrazolium bromide (MTT, Sigma); dithiothreitol (DTT, Sigma-Aldrich); and SYBR® Safe DNA gel stain, 10,000× concentrate in DMSO (Invitrogen, Carlsbad, Calif.), were purchased in the highest purity and used without further purification. The plasmids pCMV-Luc was constructed by inserting a firefly luciferase reporter gene into a pCI plasmid vector driven by the CMV promoter (Promega, Madison, Wis.). The plasmid pCMV-Luc can be amplified in E. coli DH5α and purified using a Maxiprep kit (Invitrogen) according to the manufacturer's instructions. Dulbecco's Modified Eagle's medium (DMEM), penicillin-streptomycin (P/S), fetal bovine serum (FBS), trypsin-like enzyme (TrypLE Express), and Dulbecco's phosphate buffered saline (PBS) were all purchased from Invitrogen-Gibco (Carlsbad, Calif.). Luciferase assay system with reporter lysis buffer was purchased from Promega. BCA™ protein assay system was purchased from Thermo Scientific (Rockford, Ill.). YOYO-1 iodide (1 mM solution in DMSO) was purchased from Molecular Probes (Eugene, Oreg.). Hela cells (human cervical cancer cell line) and C2C12 (mouse myoblast cell line) were purchased from the American Type Culture Collection (ATCC) and cultured according to recommended protocols.

### Example 1

The synthesis of poly(CBA-SP) is illustrated in FIG. 1. Briefly, spermine (SP, 0.202 g, 1 mmol) and 2-acetyldimedone (Dde-OH, 0.419 g, 2.3 mmol) were added into a flask and dissolved in 1 mL MeOH, stirring at room temperature (RT) for 24 h to protect primary amine groups in spermine. The next day, N,N′-cystaminebisacrylamide (CBA, 0.260 g, 1 mmol) was added into the same flask and the mixture was dissolved in 1.5 mL MeOH/diH₂O (9/1 v/v). Polymerization was conducted in an oil bath at 60° C. in the dark under a nitrogen atmosphere for 3-4 days. Then, 10% mol excess ethylenediamine (EDA) was added into reaction solution to consume any unreacted acrylamide functional groups, and the reaction was performed at 60° C. for at least additional 2 h. After that, the product was precipitated with 40 mL anhydrous diethyl ether and dried to get the intermediate polymer poly(CBA-SP-Dde). Subsequently, Dde protection groups were removed with the deprotection mixture of NH₂OH.HCl/Imidazole/NMP/DMF, stirring at room temperature for 4 h. The deprotection mixture was prepared as follows: 1.25 g (1.80 mmol) of hydroxylamine hydrochloride (NH₂OH.HCl) and 0.918 g (1.35 mmol) of imidazole were suspended in 5 mL of N-methyl-2-pyrrolidone (NMP), and the mixture was sonicated until complete dissolution; just before reaction, 5 volume of this solution was diluted with 1 volume of N,N-dimethylformamide (DMF). After deprotection, the crude product was further purified by dialysis (MWCO=1000) against MilliQ water for 24 h, followed by lyophilization to obtain poly(CBA-SP) as solid powder.

### Example 2

Poly(CBA-APPD) was prepared according to the procedure of Example 1, except that N,N′-bis(3-aminopropyl)-1,3-propanediamine (APPD) was substituted for spermine.

### Example 3

Poly(CBA-APED) was prepared according to the procedure of Example 1 except that N,N′-bis(3-aminopropyl)ethylenediamine (APED) was substituted for spermine.

### Example 4

Poly(CBA-AEPD) was prepared according to the procedure of Example 1 except that N,N′-bis(2-aminoethyl)-1,3-propanediamine (AEPD) was substituted for spermine.

### Example 5

Poly(CBA-TETA) was prepared according to the procedure of Example 1 except that and triethylenetetramine (TETA) was substituted for spermine.

### Example 6

The poly(disulfide amine)s prepared in Examples 1-5 were analyzed by ¹H NMR (400 MHZ, D₂O) and the following data were obtained.

Poly(CBA-SP): δ=3.37 (CONHCH₂CH₂SS, 4H), 2.87 (CONHCH₂CH₂SS, 4H), 2.77 (NCH₂CH₂CH₂NH₂, 4H), 2.70 (NHCOCH₂CH₂N, 4H), 2.53 (NCH₂CH₂CH₂NH₂, 4H), 2.47 (NCH₂CH₂CH₂CH₂N, 4H), 2.34 (NHCOCH₂CH₂N, 4H), 1.75 (NCH₂CH₂CH₂NH₂, 4H), 1.35 (NCH₂CH₂CH₂CH₂N, 4H).

Poly(CBA-APPD): δ=3.36 (CONHCH₂CH₂SS, 4H), 2.86 (CONHCH₂CH₂SS, 4H), 2.71 (NHCOCH₂CH₂N, 4H; NCH₂CH₂CH₂NH₂, 4H), 2.46 (NCH₂CH₂CH₂N, 4H), 2.36 (NCH₂CH₂CH₂NH₂, 4H), 2.30 (NHCOCH₂CH₂N, 4H), 1.70 (NCH₂CH₂CH₂NH₂, 4H), 1.53 (NCH₂CH₂CH₂N, 2H).

Poly(CBA-APED): δ=3.38 (CONHCH₂CH₂SS, 4H), 2.86 (CONHCH₂CH₂SS, 4H), 2.70 (NHCOCH₂CH₂N, 4H; NCH₂CH₂CH₂NH₂, 4H), 2.49 (NCH₂CH₂CH₂NH₂, 4H; NCH₂CH₂N, 4H), 2.30 (NHCOCH₂CH₂N, 2H), 1.50 (NCH₂CH₂CH₂NH₂, 4H).

Poly(CBA-AEPD): δ=3.37 (CONHCH₂CH₂SS, 4H), 2.94 (CONHCH₂CH₂SS, 4H), 2.82 (NCH₂CH₂NH₂, 4H), 2.70 (NHCOCH₂CH₂N, 4H), 2.60 (NCH₂CH₂NH₂, 4H), 2.44 (NCH₂CH₂CH₂N, 4H), 2.30 (NHCOCH₂CH₂N, 4H), 1.48 (NCH₂CH₂CH₂N, 2H).

Poly(CBA-TETA): δ=3.38 (CONHCH₂CH₂SS, 4H), 2.93 (CONHCH₂CH₂SS, 4H), 2.83 (NCH₂CH₂NH₂, 4H), 2.69 (NHCOCH₂CH₂N, 4H), 2.62 (NCH₂CH₂NH₂, 4H), 2.49 (NCH₂CH₂N, 4H), 2.30 (NHCOCH₂CH₂N, 4H).

The ¹H NMR spectra of all five of these poly(disulfide amine)s are given in FIGS. 2A-E.

### Example 7

The molecular weights of the five poly(disulfide amine)s were determined by size exclusion chromatography (SEC) on an AKTA FPLC system (Amersham Biosciences, Piscataway, N.J.) equipped with a SUPEROSE 12 column, and UV and refractive index detectors, eluted with Tris buffer (20 mM, pH 7.4) at a rate of 0.5 mL/min. Molecular weights were calibrated with standard poly[N-(2-hydroxypropyl)methacrylamide] (pHPMA). The FPLC data of these polymers are given in FIGS. 3A-E.

Table 1 shows that the range of the weight average molecular weight (M_{w}) of these polymers was 3.80 kDa to 6.12 kDa, while the range of the number average molecular weight (Mₙ) was 3.17 kDa to 4.62 kDa. The low polydispersity index (PDI=M_{w}/Mₙ), ranging from 1.15 to 1.33, indicated that these poly(disulfide amine)s have a narrow molecular weight distribution.

TABLE 1

| | | | | Buffering |
|---|---|---|---|---|
| | | | | Capacity at pH |
| Polymer | Mₙ (kDa) | M_{w} (kDa) | PDI | 7.4-5.1 (%) |
| Poly(CBA-SP) | 4.16 | 4.81 | 1.16 | 38.0 |
| Poly(CBA- | 3.36 | 4.23 | 1.26 | 36.0 |
| APPD) | | | | |
| Poly(CBA- | 4.62 | 6.12 | 1.33 | 26.0 |
| APED) | | | | |
| Poly(CBA- | 3.64 | 4.19 | 1.15 | 28.0 |
| AEPD) | | | | |
| Poly(CBA- | 3.17 | 3.80 | 1.20 | 28.0 |
| TETA) | | | | |

### Example 8

The buffering capacities of the five poly(disulfide amine)s were determined by acid-base titration. An amount equal to 5 mM of amine groups of the poly(disulfide amine)s was dissolved in 10 mL of 0.1 M NaCl aqueous solution. The pH of the polymer solutions was set initially to pH 11.0 by 0.1 M NaOH, and the solution was titrated to pH 3.0 with aliquots of 0.01 M HCl with a Corning pH meter 340. For comparison, branched PEI (M_{w}=25 kDa) was also titrated use the same method. The pH's of the solutions were measured after each addition. The buffering capacity is defined as the percentage of amine groups becoming protonated from pH 7.4 to 5.1 and can be calculated from the following equation:

Buffering capacity (%)=[(ΔV_{HCl}×0.01 M)/(Nmol)]×100

ΔV_{HCl} is the volume of 0.01 M HCl solution that brought the pH value of the polymer solution from 7.4 to 5.1, and Nmol (5 mmol) is the total moles of protonatable amine groups in the particular poly(disulfide amine). FIG. 4 shows the titration curves, and Table 1 shows the buffering capacities.

### Example 9

Polyplexes were prepared by vortexing 1 μg pDNA with each of the five poly(disulfide amine)s and bPEI 25 kDa at predetermined w/w ratios of 1, 5, 10, 20 and 30, followed by 30 min of incubation. The polyplexes were then diluted in 2 mL of dust-free diH₂O and the average particle sizes of polyplexes were measured using a BI-200SM Dynamic Light Scattering (DLS, Brookhaven Instrument Corporation, Holtsville, N.Y.) at 633 nm incident beam. Measurements were made at 25° C. at an angle of 90°. Measurements for each sample were repeated three times and reported as mean values±standard deviations (FIG. 5).

### Example 10

Agarose gel electrophoresis (1%, w/v) containing 0.5 μg/mL SYBR® Safe DNA gel stain was prepared in TAE (Tris-Acetate-EDTA) buffer. Polyplexes (0.5 μg pDNA) at w/w ratios of 0.1, 0.2, 0.5, 1, 2, 5, 10, 20 and 30 were prepared in HEPES buffer. Also, bPEI 25 kDa/pDNA complexes at a w/w ratio of 1 was prepared for comparison. The samples were mixed with 6×loading dye and the mixtures were loaded onto an agarose gel. The gel was run at 100 V for 30 min and the location of DNA bands was visualized with a UV illuminator using a Gel Documentation Systems (Bio-Rad, Hercules, Calif.). The DNA release from polyplexes was evaluated by incubating polyplexes with 10 mM DTT at 37° C. for 1 h. The samples were then analyzed by gel electrophoresis as described above (FIGS. 6A-B).

### Example 11

The family of poly(disulfide amine)s mediated transfection was evaluated on Hela cells (human cervical cancer cell line, ATCC) and C2C12 cells (mouse myoblast cell line, ATCC) using the plasmid pCMV-Luc as a reporter. Cells were maintained in DMEM containing 10% FBS, streptomycin (100 μg/mL) and penicillin (100 units/mL) at 37° C. in a humidified atmosphere with 5% CO₂. Cells were seeded 24 hrs prior to transfection in 24-well plates at initial density of 4.5×10⁴ cells/well. DNA was complexed with the poly(CBA-SP), poly(CBA-APPD), poly(CBA-APED), poly(CBA-AEPD), and poly(CBA-TETA) at w/w ratios of 1, 5, 10, 20 and 30 in HEPES buffer and incubated for 30 min before use. At the time of transfection, the medium in each well was replaced with fresh serum-free medium. Polyplexes (1.0 μg/mL DNA) were incubated with the cells for 4 h at 37° C. The medium was then replaced with 500 μL of fresh complete medium and cells were incubated for additional 44 h. The cells were then washed with pre-warmed PBS, treated with 100 μL cell lysis buffer and subjected to a freezing-thawing cycle. Cellular debris was removed by centrifugation at 16,000 rpm for 2 min. The luciferase activity in cell lysate (25 μL) was measured using a luciferase assay kit (100 μL luciferase assay buffer) on a luminometer (Dynex Technologies Inc., Chantilly, Va.). The relative luminescent unit (RLU) of luciferase expression was normalized against protein concentration in the cell extracts, measured by a BCA protein assay kit (Pierce, Rockford, Ill.). All transfection assays were carried out in triplicate (FIGS. 7A-B).

### Example 12

In vitro cytotoxicity was determined by using C2C12 cells prepared as mentioned before. DNA was complexed with the poly(CBA-SP), poly(CBA-APPD), poly(CBA-APED), poly(CBA-AEPD), poly(CBA-TETA) and bPEI at w/w ratios of 1, 5, 10, 20 and 30 in HEPES buffer and incubated for 30 min before use. Polyplexes (1.0 μg/mL DNA) were incubated with the cells for 4 hrs in serum-free medium followed by 20 hrs in complete medium. MTT solution (50 μL, 2 mg/mL) was then added and cells were further incubated for 2 hrs. The medium was removed and 300 μL DMSO was then added to each well. The absorption was measured at 570 nm using a microplate reader (Model 680, Bio-Rad Lab, Hercules, Calif.). The percentage relative cell viability was determined relative to control (untreated) cells, which were not exposed to transfection system and taken as 100% cell viability. All cytotoxicity experiments were performed in triplicate (FIG. 8).

### Example 13

The cellular uptake of polyplexes was examined by flow cytometry. Approximately 2×10⁵ C2C12 cells were seeded in 12-well plates 24 h prior to study. YOYO-1 idodide-tagged pCMV-Luc (1 molecule or YOYO-1 dye per 20 base pairs of nucleotide) was prepared 30 min before use. Then, polyplexes were prepared by mixing poly(disulfide amine)s with YOYO-1-labeled plasmid DNA as w/w ratios of 1, 5, 10, 20, and 30, as described above. At the time of transfection, fluorescence-labeled polyplexes (1.0 μg/mL DNA) was incubated with cells at 37° C. for 4 h in serum-free medium. Then, medium was removed by aspiration, and the cells were washed with PBS, harvested by trypsin-like enzyme (TrypLE Express), and neutralized with serum containing medium. After centrifugation at 1500 rpm for 2 min, cells were fixed with 2% paraformaldehyde in PBS-0.5% BSA solution for 20 min at room temperature, and washed and resuspended in 0.3 mL ice-cold PBS. Samples were kept in ice and analyzed by a FACScan analyzer (BD Biosciences, San Jose, Calif.) at a minimum of 1×10⁴ cells using FL1-height channel for YOYO-1 dye. Data were analyzed with Windows Multiple Document Interface software, version 2.9 (WinMDI, Microsoft, Redmond, Wash.). Results are shown in FIGS. 9A-E.

1-34. (canceled)

35: A composition comprising a biodegradable polydisulfide amine selected from poly(N,N-cystaminebisacrylamide-spermine), poly(N,N′-cystaminebisacrylamide-N,N′-bis(3-aminopropyl)-1,3-propanediamine), poly(N,N′-cystaminebisacrylamide-(3-aminopropyl)ethylenediamine), poly(N,N′-cystaminebisacrylamide-N,N′-bis(2-aminoethyl)-1,3-propanediamine), and poly(N,N′-cystaminebisacrylamide-triethylenetetramine).

36: The composition of claim 35 wherein the polydisulfide amine comprises poly(N,N′-cystaminebisacrylamide-spermine).

37: The composition of claim 35 wherein the polydisulfide amine comprises poly(N,N′-cystaminebisacrylamide-N,N′-bis(3-aminopropyl)-1,3-propanediamine).

38: The composition of claim 35 wherein the polydisulfide amine comprises poly(N,N′-cystaminebisacrylamide-N,N′-bis(3-aminopropyl)ethylenediamine).

39: The composition of claim 35 wherein the polydisulfide amine comprises poly(N,N′-cystaminebisacrylamide-N,N′-bis(2-aminoethyl)-1,3-propanediamine).

40: The composition of claim 35 wherein the polydisulfide amine comprises poly(N,N′-cystaminebisacrylamide-triethylenetetramine).

41: A complex comprising a selected nucleic acid bonded to a biodegradable polydisulfide amine selected from poly(N,N′-cystaminebisacrylamide-spermine), poly(N4V-cystaminebisacrylamide-N,N′-bis(3-aminopropyl)-1,3-propanediamine), poly(N,N′-cystaminebisacrylamide-N,N′-bis(3-aminopropyl)ethylenediamine), poly(N,N′-cystaminebisacrylamide-N,N′-bis(2-aminoethyl)-1,3-propanediamine), and poly(N,N′-cystaminebisacrylamide-triethylenetetramine).

42: The complex of claim 41 wherein the polydisulfide amine comprises poly(N,N′-cystaminebisacrylamide-spermine).

43: The complex of claim 41 wherein the polydisulfide amine comprises poly(N,N′-cystaminebisacrylamide-N,N′-bis(3-aminopropyl)-1,3-propanediamine).

44: The complex of claim 41 wherein the polydisulfide amine comprises poly(N,N′-cystaminebisacrylamide-N,N′-bis(3-aminopropyl)ethylenediamine).

45: The complex of claim 41 wherein the polydisulfide amine comprises poly(N,N′-cystaminebisacrylamide-N,N′-bis(2-aminoethyl)-1,3-propanediamine).

46: The complex of claim 41 wherein the polydisulfide amine comprises poly(N,N′-cystaminebisacrylamide-triethylenetetramine).

47: The complex of claim 41 wherein the selected nucleic acid comprises a plasmid.

48: The complex of claim 41 wherein the selected nucleic acid comprises siRNA.

49: The complex of claim 41 wherein the selected nucleic acid comprises an oligonucleotide.

50: A method for transfecting mammalian cells, the method comprising contacting selected mammalian cells with a complex comprising a nucleic acid bonded to a polydisulfide amine selected from poly(N,N′-cystaminebisacrylamide-spermine), poly(N,N′-cystaminebisacrylamide-N,N′-bis(3-aminopropyl)-1,3-propanediamine), poly(N,N′-cystaminebisacrylamide-N,N′-bis(3-aminopropyl)ethylenediamine), poly(N,N′-cystaminebisacrylamide-N,N′-bis(2-aminoethyl)-1,3-propanediamine), and poly(N,N′-cystaminebisacrylamide-triethylenetetramine).

51: The method of claim 50 wherein the selected nucleic acid comprises a plasmid.

52: The method of claim 50 wherein the selected nucleic acid comprises siRNA.

53: The method of claim 50 wherein the selected nucleic acid comprises an oligonucleotide.
